(19) United States
(12) Patent Application Publication
(10) Pub. No.: US 2015/0105698 A1
(43) Pub. Date: Apr. 16, 2015

(54) METHOD FOR KNEE RESECTION ALIGNMENT APPROXIMATION IN KNEE REPLACEMENT PROCEDURES

(71) Applicant: Somersault Orthopedics Inc., Pleasanton, CA (US)

(72) Inventor: Ilwhan Park, Walnut Creek, CA (US)

(73) Assignee: Somersault Orthopedics Inc., Pleasanton, CA (US)

(21) Appl. No.: 14/516,298

(22) Filed: Oct. 16, 2014

Related U.S. Application Data
(60) Provisional application No. 61/961,541, filed on Oct. 16, 2013.

(51) Int. Cl. A61B 17/17; G06T 7/00; A61B 5/107; A61B 5/055; A61B 5/11

(52) U.S. Cl. CPC A61B 17/1703; A61B 5/055; A61B 5/1121; A61B 5/1079; G06T 7/0044; G06T 7/0085; G06T 7/0097; G06T 2207/20101; G06T 2207/30008; G06T 2207/10088
USPC 600/587; 382/131

(57) ABSTRACT

Aspects of the present disclosure involve systems, methods, computer program products, and the like, for utilizing a series of images of a patient's anatomy to determine a cut plane for use during a knee procedure. To determine a cut plane for use during a knee replacement procedure, the 2D images may be analyzed by a computer program to determine a femoral shaft axis reference line, a center of rotation of the knee, and the valley floor of the condyle notch. With these landmarks identified in the images, a cut plane through the femur for use during a TKA procedure may be determined. Further, the location of these features in the images may be determined by analyzing the gray scale value of one or more pixels around a selected point on the image. The pixel with the lowest gray scale value may then be assumed to be the edge of the cortical bone in the 2D image.

### CROSS-REFERENCE TO RELATED APPLICATIONS

This application claims priority under 35 U.S.C. §119(e) to U.S. Provisional Application No. 61/961,541 entitled “IMPROVEMENTS IN KNEE FEMUR REPLACEMENT AND ALIGNMENT”, filed on Oct. 16, 2013 which is incorporated by reference in its entirety herein.

### TECHNICAL FIELD

Aspects of the present disclosure generally relate to systems and methods for an accurate determination of relevant dimensions and alignments (lengths, angles, etc.) associated with a procedure for partial or total replacement of a knee component of a patient. Additional aspects of the present disclosure generally relate to systems and methods for identifying a cortical bone edge in a two-dimensional image of a knee component of a patient.

### BACKGROUND

Through over-use, traumatic events and/or debilitating disease, a person's joint may become damaged to the point that the joint is repaired. One type of procedure to address damage to a person's joint is an arthroplasty procedure. Arthroplasty is a medical procedure where a joint of a patient is replaced, remodeled, or realigned, often done to relieve pain in the joint after damage. Damage to the joint may result in a reduction or wearing away of cartilage in the joint area, which operates to provide frictional, compressive, shear, and tensile cushioning within the joint. As such, reduction in cartilage in a joint causes pain and decreased mobility of the joint. To combat this joint pain, a patient may undergo the arthroplasty procedure to restore function and use back to the damaged joint.

One type of arthroplasty procedure is known as Total Knee Arthroplasty (TKA). In general, TKA involves replacing the diseased or damaged portion of the knee with metal or plastic components that are shaped to approximate the shape of the replaced portion or shaped to allow movement of the joint and relieve the joint pain. Thus, a TKA procedure may include replacement of a portion of the femur and a portion of the tibia that make up the knee joint. Similar procedures may be performed on other damaged joints, such as a hip, a shoulder, an elbow, and the like. General discussion of arthroplasty procedures herein are directed specifically to TKA-type procedures, but may be applied to arthroplasty procedures of other types of joints.

In a TKA procedure, a damaged portion of the distal region of the femur is removed and replaced with a metal or plastic component that is shaped to mirror or approximate the replaced portion. The metal or plastic component may be impacted onto the femur or fixed using a type of surgical cement or other fastening system. Further, a proximal portion of the tibia may also be removed and replaced with a generally flat metal or plastic component that is shaped to mirror or approximate the replaced portion. The tibia replacement implant may also be attached to the tibia through impaction onto the bone or fixed using a type of cement. In general, the femur implant and the tibia implant are mated to form a joint that approximates the shape and operation of the knee joint. In some examples, a plastic surface is placed between the femur implant and the tibia implant to prevent metal-on-metal interaction between the implants during use of the replaced joint.

As mentioned above, a TKA procedure often involves the removal and replacement of portions of the femur and/or tibia of the injured knee. During the removal, the portions of the femur and tibia may be cut, drilled, resurfaced, and the like to create a surface on the bones that mates with the respective implants. In one particular example, the ends of the bones (distal end of the femur and proximate end of the tibia) may be completely removed to create a generally flat surface to which the implants are mated. Once the mating surfaces for the implants are created on the receiving bones, the implants may then be attached to the bones as described above.

Although the broad outline of the TKA procedures is described above, there is much to consider when performing the procedure. For example, patients may undergo a preoperative planning phase of the procedure through one or more consultations with a doctor that could last a month or more before the TKA is performed. In addition, alignment of the implants in the joint with the rest of the patient's anatomy is crucial to the longevity of the implant and the implant's effectiveness in counteracting the pre-TKA joint condition. As such, systems and methods have been developed to produce customized arthroplasty cutting jigs that allow a surgeon to quickly and accurately perform the necessary resections of the bones that result in a successful TKA procedure. In particular, cutting jigs may be generally customized for the particular patient's joint undergoing the TKA procedure to ensure that the implants align with the patient's anatomy post-procedure. Through the use of such customized cutting jigs, the TKA procedure is both more accurate (ensuring more longevity to the implants) and quicker (reducing the time required for the surgical procedure, thereby reducing the potential for post-surgery complications).

In general, cutting guides or cutting jigs used in TKA procedures may attach to one or more bones of the knee and provide a cut line to the surgeon for use during the TKA surgery. In particular, a femur cutting jig may attach to the distal end of the femur and include a cut guide or line. A surgeon, during the procedure, inserts a saw device into or through the cut line to resect the distal end of the femur. Similarly, a tibia cutting jig may attach to the proximal end of the tibia and include a cut line that the surgeon uses to resect the proximal end of the tibia. In this manner, the ends of the femur and tibia are resected by the surgeon during the TKA procedure, thereby creating a smooth mating surface for the implants. As should be appreciated, the location and angle of the cut plane through the respective bone surface indicated by the cutting jig may determine the overall effectiveness of the TKA procedure. As such, a cutting jig utilized during the procedure should be designed to provide the proper location and orientation of the cut plane on the bones of the affected joint such that treatment of the region can be performed accurately, safely, and quickly.

It is with these and other issues in mind that various aspects of the present disclosure were developed.

### SUMMARY

One implementation of the present disclosure may take the form of a system for processing a medical scan of a patient. The system comprises a network interface configured to receive one or more medical images of a patient's anatomy, a processing device in communication with the network interface, and a computer-readable medium in communication with the processing device configured to store information and instructions. Several operations are performed when the information and instructions are executed by the processing device. In particular, the operations of receiving the one or more medical images of the patient's anatomy from an imaging device, obtaining a reference point on at least one of the medical images, the at least one medical image comprising a plurality of pixels, and associating the reference point to a reference pixel from the plurality of pixels of the at least one medical image. Further operations include creating a first range of pixels from the plurality of pixels of the at least one medical image corresponding to the reference pixel, wherein each pixel in the first range of pixels comprises a gray scale value, determining the pixel in the first range of pixels with the lowest gray scale value, and setting the determined pixel in the first range of pixels with the lowest gray scale value as a location of a bone edge in the at least one medical image.

Another implementation of the present disclosure may take the form of a method for determining a cut plane through a human femur during an arthroplasty procedure on a human knee. The method may include the operations of receiving a plurality of two-dimensional (2D) images of a patient's joint subject to the arthroplasty procedure at a computing device, calculating a center of rotation of the human knee based at least on the one or more locations within at least one of the plurality of 2D images corresponding to a posterior base of a plurality of condyles of the human femur, and determining a best fit plane for a plurality of valley points along a valley floor of a trochlear groove of the human femur of a first set of 2D images of the plurality of 2D images. The method may also include the operations of calculating a cut plane for use during an arthroplasty procedure on a human knee, wherein the cut plane is parallel to the calculated center of rotation of the human knee and perpendicular to the best fit plane for the identified plurality of valley points along a valley floor of a trochlear groove of the human femur and generating a cutting jig for the arthroplasty procedure comprising a cut slot corresponding to the calculated cut plane.

Yet another implementation of the present disclosure may take the form of a method for determining a resection plane of a femur of a human patient. The method may include the operations of receiving a plurality of two-dimensional (2D) images of a patient's knee at a computing device from an imaging device, the 2D images comprising a femur of the patient's knee, identifying one or more locations within at least one of the plurality of 2D images, the one or more locations corresponding to the posterior base of the plurality of condyles of the femur, and calculating a center of rotation axis of the patient's knee based at least on the one or more locations within at least one of the plurality of 2D images corresponding to a posterior base of a plurality of condyles of the human femur. Further, the method may include the operations of utilizing the center of rotation axis of the patient's knee to determine a cut plane through the femur during a resection of the femur for a arthroplasty procedure on the patient's knee and generating a cutting jig for the arthroplasty procedure comprising a cut slot corresponding to the cut plane.

### BRIEF DESCRIPTION OF THE DRAWINGS

FIG. 1 is an anterior view of a knee joint illustrating the femur, tibia and ligaments of the joint.

FIG. 2A is an anterior view of a lower femur.

FIG. 2B is a posterior view of a lower femur.

FIG. 3 is a flowchart illustrating a method for locating a cortical bone edge in a 2D image of a patient's femur.

FIG. 4 is an illustration of one embodiment for obtaining 2D images of a knee of a patient.

FIG. 5 is a screenshot of a magnetic resonance imaging (MRI) image of a patient's femur.

FIG. 6 is a screenshot of a close-up view of an MRI image of a patient's femur with a selected point and a horizontal pixel range around the selected point.

FIG. 7 is a chart illustrating gray scale values of the pixels in the pixel range of the MRI image of FIG. 6.

FIG. 8 is a screenshot of the MRI image of the patient's femur with a selected point and a vertical pixel range around the selected point.

FIG. 9 is a screenshot of the MRI image of the patient's femur with a plurality of horizontal pixel ranges extending from a selected point by a set distance value.

FIG. 10 is a flowchart illustrating a method for determining a cut plane for use during a knee replacement femur from one or more 2D images of the knee.

FIG. 11 is an illustration of a perspective view of a lower portion of a femur shaft component.

FIG. 12 is an illustration of a sagittal view of a lower portion of a femur indicating a center of rotation of the knee.

FIG. 13 illustrates a sequence of curves representing MRI slices of the femur in FIG. 12 in a sagittal view.

FIG. 14 illustrates a condyle notch valley floor for a condyle notch in the images of a patient's femur.

FIG. 15 is a block diagram illustrating an example of a computing device or computer system which may be used in implementing the embodiments disclosed above.

### DETAILED DESCRIPTION

Aspects of the present disclosure involve systems, methods, computer program products, manufacture process and the like, for utilizing a series of images of a patient's anatomy to determine a cut plane for use during a knee procedure. In particular, the present disclosure provides for a method of utilizing one or more two-dimensional (2D) images of the patient's joint to undergo an arthroplasty procedure. The method includes receiving the 2D images of the joint from an imaging device and determining the location within at least one of the 2D images of the patient's cortical bone edge. In general, the location of the cortical bone edge of the patient's knee is determined by analyzing the gray scale value of one or more pixels around a selected point on the image. In particular, a range of pixels around the selected point provides a range of gray scale values that may be analyzed to determine the pixel with the lowest gray scale value. This pixel may then be assumed to be the edge of the cortical bone in the 2D image.

To determine a cut plane for use during a knee replacement procedure, the 2D images may be analyzed by a computer program or a user of a computing device to determine several landmarks or aspects of the patient's knee anatomy. In one particular example, one or more of the landmarks or aspects may be determined through the edge detection of the cortical bone described above. For example, the computing device may determine a femoral shaft axis reference line. Further, a center of rotation around which the femur, tibia and trochlear groove move during articulation of the knee may be determined. Also, a valley floor condyle notch reference line or reference plane may also be determined from the images of the patient's knee from which a normal to the reference plane may be calculated. With the femoral shaft axis line, reference plane, and the center of rotation of the knee identified, a cut plane through the femur for use during a TKA procedure may be determined that is perpendicular to the normal of the reference plane and parallel to the center of rotation. The depth of the cut plane on the femur may be determined by the type of implant selected for the procedure. Through the methods described herein, a reliable and sturdy cut plane for purposes of a knee implant may be determined. Further, the procedure to determine the cut plane through the femur does not require the generation of a 3D model of the patient's knee so that the TKA procedure may occur more quickly and efficiently than previous procedure method.

To aid in the description below of the customized arthroplasty cutting jigs and methods for creating said jigs, a brief discussion of the bone anatomy of the human knee is now included. As mentioned above, the present disclosure may be applied to any type of joint of a patient. However, for ease of understanding, the discussion herein is limited to particulars of the human knee as an example of the joint relating to the present disclosure procedure and apparatus.

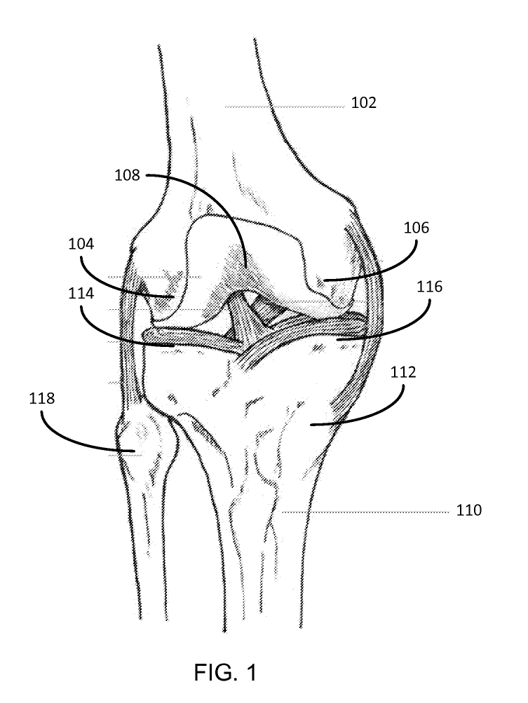

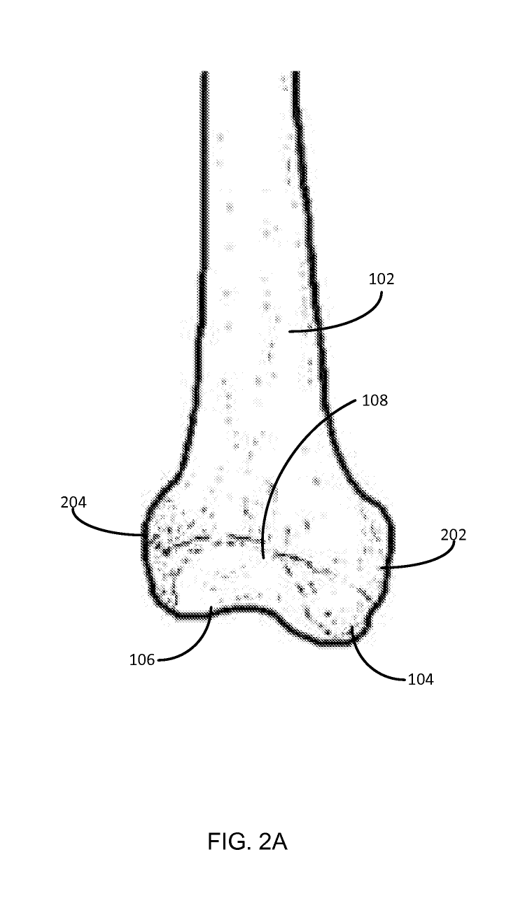

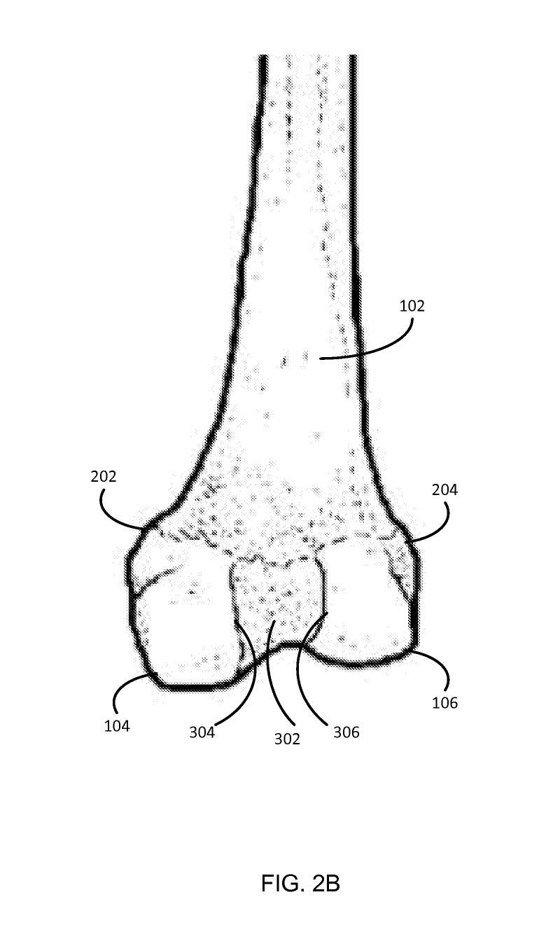

FIG. 1 illustrates an anterior view of a patient knee joint, and in particular, the femur, tibia, and ligaments of the knee. The femur is proximal the tibia and includes two eminences, known as the condyles 104,106. Between the condyles is a smooth depression called the trochlea 108 or trochlear groove. The condyles are divided into a medial condyle 104 and a lateral condyle 106. The tibia 110 includes a head with two tuberosities, a medial tuberosity 114 and a lateral tuberosity 116. The medial 114 and lateral tuberosity 116 generally form concave surfaces (known as the tibia plateau) in the head 112 of the tibia 110. In general, the condyles 104,106 form two convex surfaces that engage and articulate with two convex surfaces of the tuberosities 114,116 of the tibia 110 during operation of the knee joint. A fibula bone 118 is also shown in FIG. 1 that attaches to the tibia 110 at or near the tibia head 112. Additional features and details of the femur 102 and the tibia 110 of the knee joint are discussed in more detail below with reference to FIGS. 2 through 5.

FIG. 2A is an anterior view of the lower or distal end of the femur illustrating the portions of the femur associated with the knee joint. Thus, similar to the discussion above with reference to FIG. 1, the femur 102 includes a medial condyle 104 and a lateral condyle 106. Between the condyles is the trochlea 108. The outer surface of the medial condyle 104 is a small eminence known as the outer or medial tuberosity 202 which provides an attachment area for external lateral ligaments of the knee. Similarly, the outer surface (with reference to the center of the bone) of the lateral condyle 106 is an eminence known as the inner or lateral tuberosity 204 which provides an attachment area for the internal lateral ligaments.

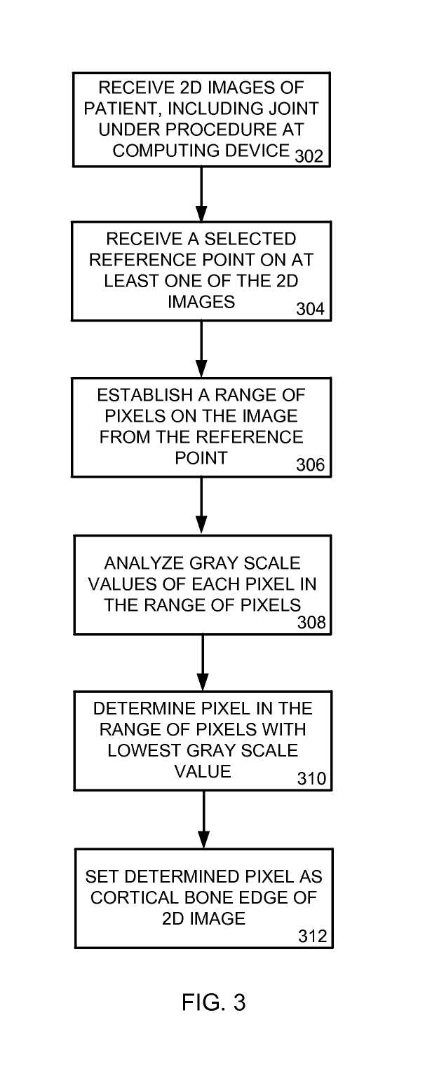

Turning now to the posterior view of the lower femur illustrated in FIG. 2B, the posterior views of the medial condyle 104 and the lateral condyle 106 are shown. Between the condyles 104,106 of the posterior portion of the femur 102 lies a notch known as the intercondyloid notch 302. In particular, the inner surfaces 304, 306 of the posterior portions of the medial condyle 104 and the lateral condyle 106 form the surfaces of the intercondyloid notch 302. Also shown in FIG. 3 is the posterior view of the medial tuberosity 202 and the lateral tuberosity 204 along the outer surfaces of the medial condyle 104 and the lateral condyle 106, respectively.

In general, during a TKA procedure, portions of the distal end of the femur (such as that shown in FIGS. 2 and 3) are removed by the surgeon and replaced with an implant that approximates the shape and function of the ends of the respective bones. To aid in resecting portions of the femur and tibia, the surgeon may employ a femur cutting jig and tibia cutting jig that provides a cut or resection line for the surgeon to cut along. The cut plane provided by the cutting jig may be determined based on one or more landmarks or features of a patient's anatomy, such as the edge of the femur bone, illustrated in an image of the patient's joint. Thus, it may be beneficial for determining the cut plane for the TKA procedure to accurately identify the cortical bone, or outer shell, of the patient's femur from one or more image slices of the patient's knee. One method for locating a cortical bone edge in a 2D image of a patient's femur is described in the flowchart of FIG. 3. Although more or fewer operations may be included in the process of detecting the cortical bone of the femur, the operations of FIG. 3 provide a general outline of one such process that utilizes 2D images of the patient's joint.

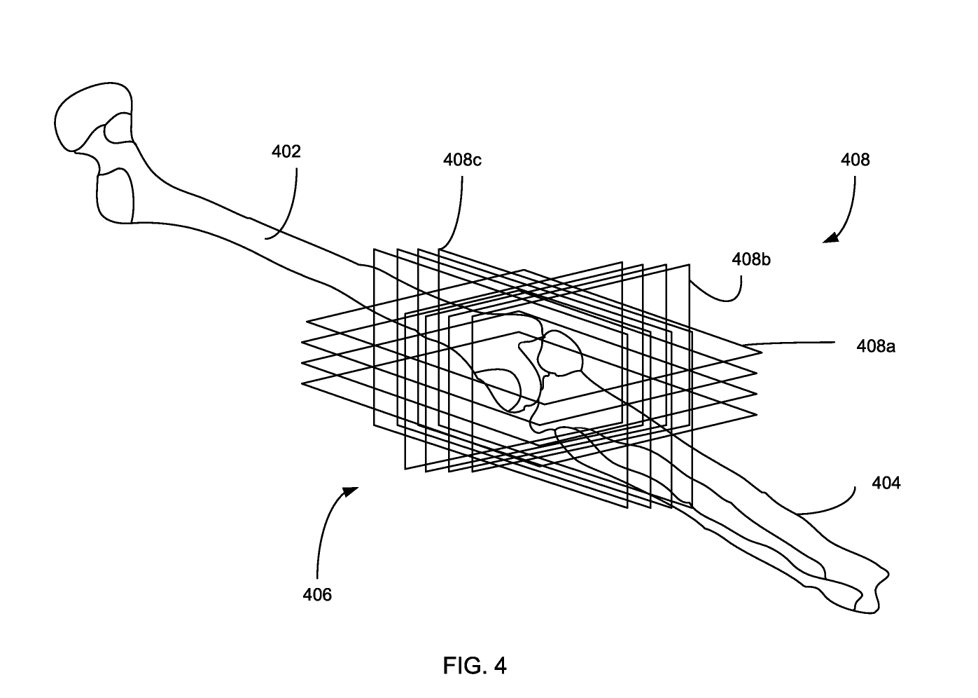

Beginning in operation 302, a series of two-dimensional (2D) images of the patient's joint on which the arthroplasty procedure is to be performed may be obtained. The 2D images of the patient's joint may be obtained from an imaging device (such as an X-ray or magnetic resonance imaging (MRI) machine) from several aspects of the joint. For example, FIG. 4 illustrates one embodiment for obtaining 2D images of a knee 406 of a patient. In particular, the patient's knee 406, including portions of the femur 402 and tibia 404, is scanned in a MRI knee coil to generate a plurality of 2D knee coil MRI images of the patient's knee. In one embodiment, the 2D images 408 of the knee include a plurality of images taken along a coronal plane 408a through the knee, a plurality of images taken along an axial plane 408b through the knee, and/or a plurality of images taken along a sagittal plane 408c through the knee. In other embodiments, the 2D images may be any combination of coronal, sagittal and/or axial views. In one embodiment, the MRI imaging spacing for the 2D knee coil images may range from approximately 2 mm to approximately 6 mm and may vary from aspect to aspect. For example, the coronal image slices 408a may be spaced 2 mm apart, while the axial image slices 408b may be spaced 6 mm apart.

While the embodiments herein are discussed in the context of the imaging being via an MRI machine, in other embodiments the imaging is via computed tomography (CT), X-ray, or other medical imaging methods and systems. Further, although it is discussed herein as a scan of the knee, the 2D images may be obtained for any joint or other area of the patient's body, such as images of the patient's ankle, hip, shoulder, etc.

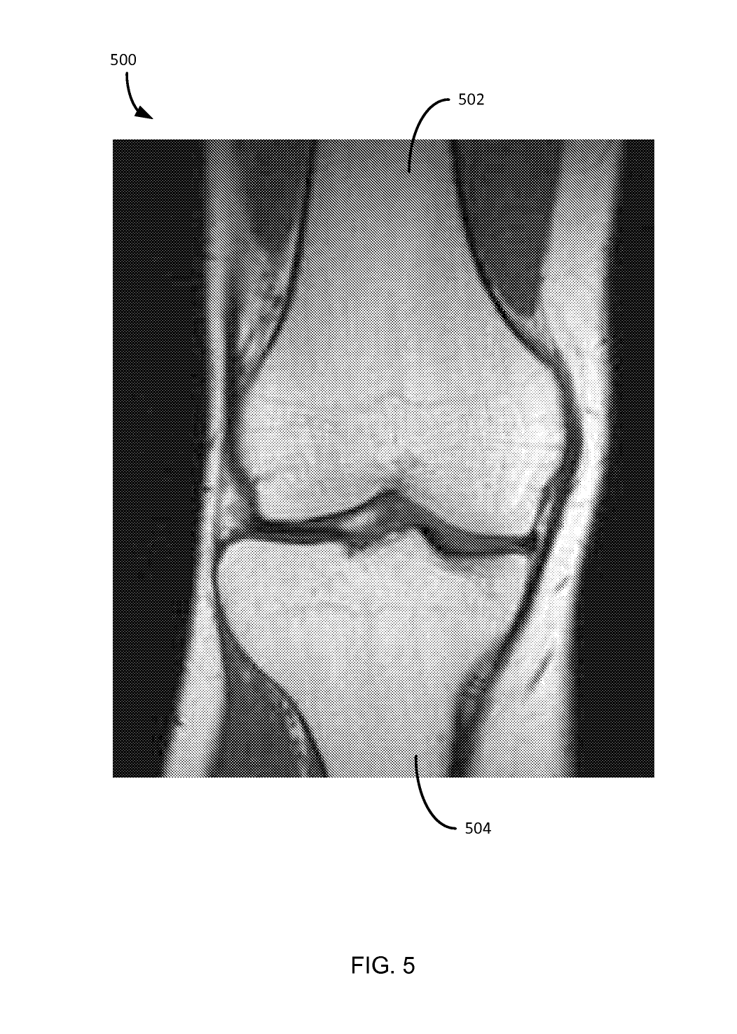

Once the 2D images of the joint at issue are obtained, the images may be received at and entered into a computing device for processing. The computing device may receive the images through any form of electronic communication with the imaging device. In one particular example, the 2D images may be obtained by the imaging device (such as the MRI imaging machine) and transmitted to a website accessible by the computing device. In general, however, the 2D images may be obtained from the imaging machine in any fashion for further processing by the computing device. One example of such an MRI image of a patient's knee is illustrated in the screenshot of FIG. 5. In particular, the MRI image 500 is a coronal image of a patient's knee illustrating the femur 502 and the tibia 504 of the knee joint. Although the MRI image 500 of FIG. 5 is referred to for the discussion herein, it should be appreciated that any type of coronal, sagittal, or axial image may be utilized.

In operation 304, the computing device may receive a selected reference point in at least one of the 2D images. To provide the reference point in one embodiment, an operator of the computing device may sit at a monitor or other interface of the computing device through which the images are viewed. Utilizing a software program executed by the computing device, the operator may view the 2D images and provide the one or more reference markers on at least one of the 2D images. These electronic markers may correspond to one or more reference points within the images for use by the computing device to determine a cortical bone portion of the bone illustrated in the 2D image. The operations to utilize the reference points to determine the bone edge or the cortical bone in the image are described in more detail below.

In another embodiment, a program executed by the computing device may obtain the 2D images and determine the one or more reference points within the images, with or without the aid of an operator of the computing device. For example, the computing device may analyze the 2D images and determine a first reference point within the image corresponding to near a presumed cortical bone surface of the bones in the image. In yet another embodiment, one or more of the operations of the method of FIG. 3 are performed by the operator, while other operations are performed by the computer program. For example, a program executed by the computing device may instruct a user of the device to locate a reference point in a particular area of the image by requesting the user to indicate the reference point near what the user may presume to be the cortical bone edge in the image. In another example, the program may analyze the 2D image to locate a potential area in the image that may include the cortical bone of the image and instruct the user to select a reference point within the potential area near a perceived cortical bone feature. As such, any of the operations and methods described herein may be performed by an operator of the computing device or the computing device itself through hardware, software, or a combination of both hardware and software.

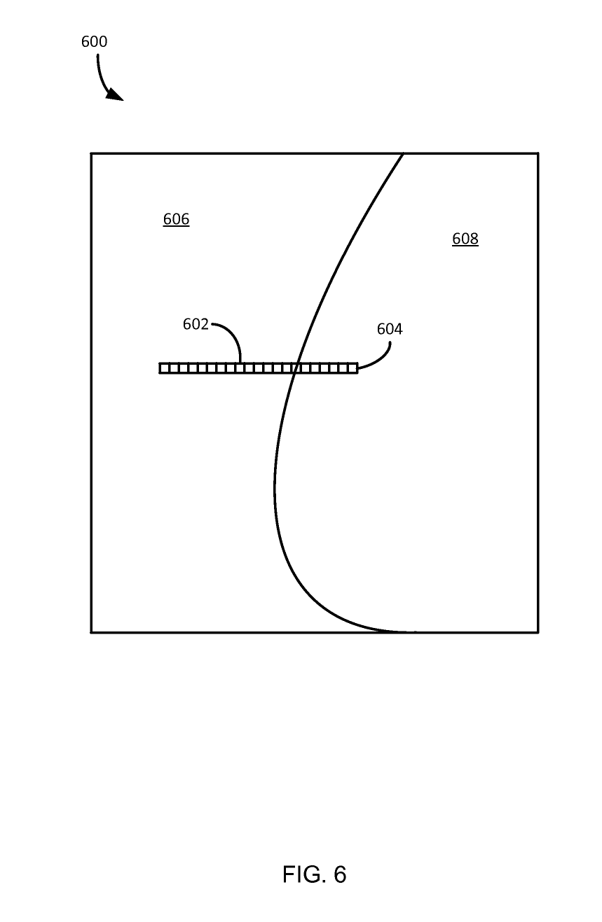

FIG. 6 is a screenshot of a close-up view of an MRI image of a patient's femur with a selected point and a horizontal pixel range around the selected point. The image 600 illustrates a non-bone region 606 and a bone region 608 of the patient. For example, the image 600 may represent a small portion of the MRI image 500 shown in FIG. 5 of the patient's knee joint. The image 600 of FIG. 6 illustrates a portion of that image that includes a region 606 illustrating portions of the patient's knee that does not include the image of the femur and a region 608 illustrating portions of the patient's knee that includes the image of femur bone. In one embodiment, the transition from the non-bone region 606 to the bone region 608 indicates the edge of the patient's femur, or the cortical bone of the patient's femur in the image 600.

Also shown in the image 600 of FIG. 6 is a reference point 602. As mentioned above, the reference point 602 may be indicated in the image by the user through operation of the computing device. Thus, the user may analyze the image and select a point on the image at or near the cortical bone of the femur. In another embodiment, the computing device may analyze the image and select a point that is at or near the cortical bone feature of the femur in the image. Regardless of the embodiment utilized, it should be appreciated that it is not required that the reference point be at the cortical bone edge in the image 600. Rather, the reference point may be in any position within the image, as discussed in more detail below.

Returning to the flowchart of FIG. 3, in operation 306 the computing device may establish a range of pixels 604 in the 2D image around the reference point 602. In the embodiment illustrated in FIG. 6, the range of pixels 604 includes pixels along the same horizontal axis of the reference point 602. In particular, the computing device associates the selected reference point 602 to a particular pixel of the image, referred to herein as the reference pixel. In the embodiment shown, the pixel range 604 is the pixels of the image on either side of the reference pixel 602 in the same horizontal axis of the image as the reference pixel. For example, the pixel range 604 may include the adjacent ten pixels to the left of the reference pixel and the adjacent ten pixels to the right of the reference pixel. That is, the pixel range 602 includes a horizontal row of pixels of the image 600 of twenty-one pixels (the reference pixel, ten pixels to the left of the reference pixel, and ten pixels to the right of the reference pixel). The particular row of the image of the range of pixels 602 is determined from the selected reference point or reference pixel 602 in the image.

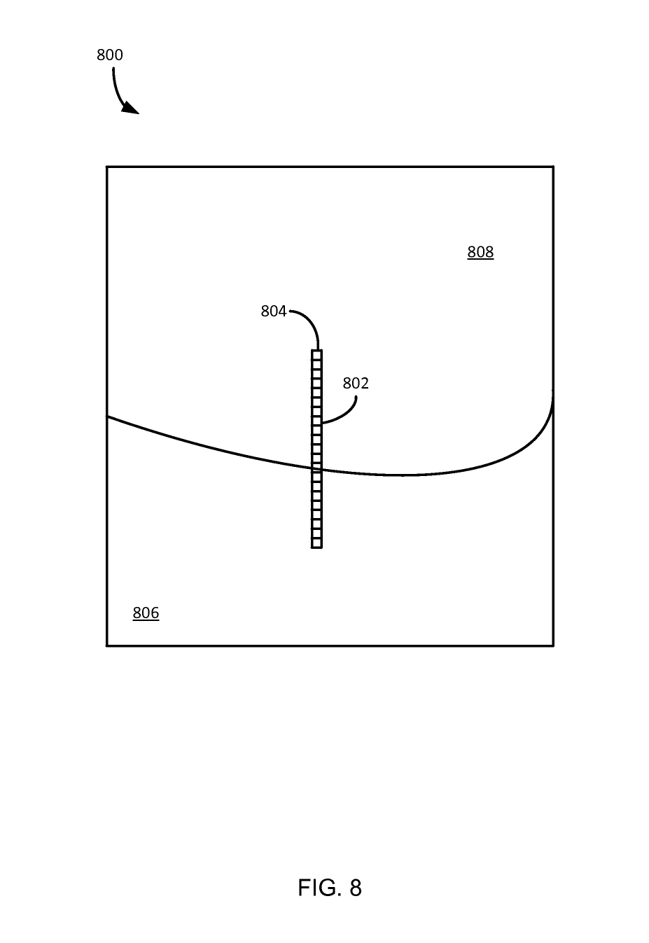

As should be appreciated, the embodiment illustrated in the image 600 is but one example of the range of pixels 604 utilized by the computing device. In another embodiment, the range of pixels may be a vertical range of pixels that extend up and down the image from the reference pixel 602. An example of a vertical range of pixels is discussed in more detail below with reference to FIG. 8. In another embodiment, the range of pixels 604 may include a combination of pixels within the same row and same column as the reference pixel 602. In yet another embodiment, the range of pixels 604 may include pixels not in the same row and/or same column as the reference pixel 602, or a combination of pixels in the same row and/or the same column as the reference pixel and pixels not in the same row and/or same column. Further, the range of pixels 604 may not be adjacent to each other within the range such that spaces between the pixels of the range may be present. Also, the range of pixels 604 may include any number of pixels. For example, it is not required that the range of pixels 604 illustrated in FIG. 6 include 21 pixels. Rather, the range 604 may include any number of pixels in the same row of the image as the reference pixel 602. As also discussed in more detail below, the number of pixels in the range of pixels 604 may be selected to increase the likelihood that the cortical bone edge in the image is located within the range. In general, the range of pixels 604 around the selected reference pixel 602 may include any number of pixels in any relation to the reference pixel.

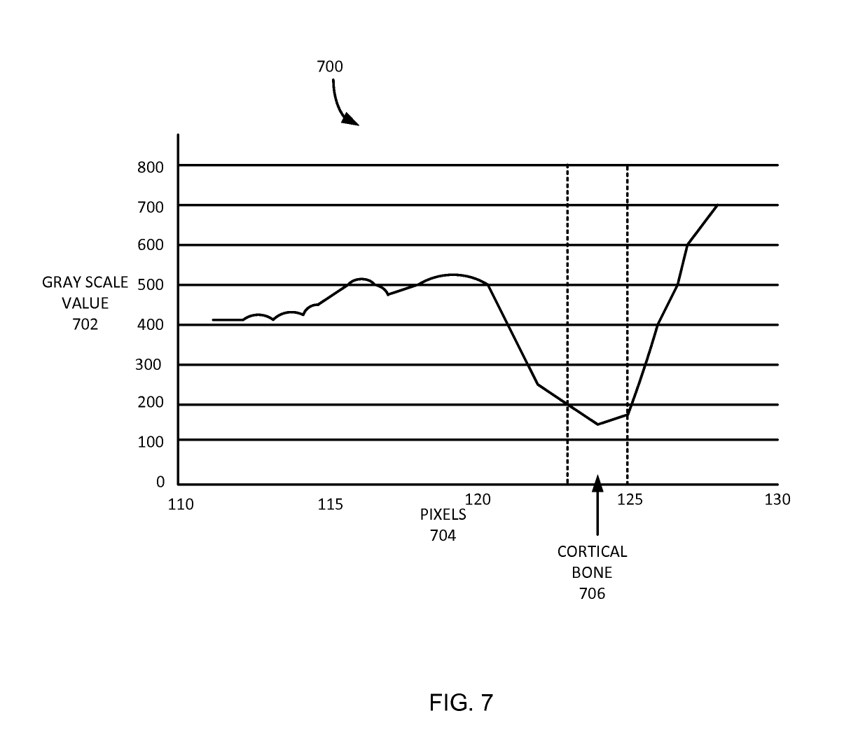

With the range of pixels 604 for analysis established, the computing device may analyze the gray scale value associated with one or more of the pixels in the range of pixels. FIG. 7 is a chart illustrating the gray scale values of the pixels in the pixel range 604 of the MRI image 600 of FIG. 6. Although shown in FIG. 7 as a chart of the gray scale values of the pixels in the pixel range, it should be appreciated that such a chart may not be created by the computing device. Rather, the computing device may simply analyze the gray scale values associated with one or more of the pixels in the pixel range 604 and determine the lowest value of gray scale in the range. However, for simplification of the discussion herein, reference is made to the chart of FIG. 7.

The chart 700 includes an x-axis of gray scale values of the pixels in the image and a y-axis of a reference number assigned to the pixels in the pixel range 604. In the example shown, the pixels of the pixel range are assigned a reference number from 110 to 130. The reference numbers assigned to the pixels may be associated with the placement of the pixels within the pixel range 604. For example, pixel number 110 may be the leftmost pixel in the pixel range and pixel number 130 may be the rightmost pixel. A similar convention may be used for a vertical pixel range such that the lowest reference number may be assigned to lowest pixel in the vertical pixel range and the highest reference number may be assigned to highest pixel in the vertical pixel range. In general, any type of reference number may be used to index the pixels in the pixel range 604. In one particular example, the pixels in the image are assigned a pixel number by the computing device that is universal to the image and the reference number in the chart may be associated or the same as the pixel number assigned by the computing device.

As shown in the chart 700, the gray scale values 702 for each of the pixels 704 in the pixel range 604 are graphed. In operation 310 of the flowchart of FIG. 3, the computing device may analyze the gray scale values 702 of the pixels 704 in the pixel range to locate the pixel or pixels with the lowest gray scale value. In the graph 700, the lowest gray scale value 706 occurs at or about pixel 124. Once the pixel with the lowest gray scale value is determined by the computing device, the computing device may then associate the location of the pixel with the lowest gray scale value in the range of pixels as the cortical bone edge of the image in operation 312. In general, the transition in the image from a darker region to a lighter region may indicate the cortical bone edge in the image. Thus, the location of the pixel in the pixel range 604 with the lowest gray scale value indicates the cortical bone edge in the accompanying image.

In another embodiment, the computing device may be configured to not only identify the pixel with the lowest gray scale value, but may also verify that the gray scale values along the pixel range provide a valley shape to the graph. The valley shape provides a stronger indication that the cortical bone edge is located at the lowest point within the valley as the gray scale values transition from a dark region to a light region and back to a dark region along the pixel range. Such a valley suggests the cortical bone edge in the image resides in the valley portion of the gray scale value chart 700. In particular, as the coordinate x 704 increases in the graph 700, the gray scale intensity of pixels within the range of pixels tends to decrease to a lowest number, corresponding to a highest bone density, then increase beyond that point. Further, in some embodiments the computing device may indicate more than one pixel in the range as being associated with the cortical bone edge in the image. In this embodiment, a group of pixels may be designated as providing the cortical bone edge such that the computing device may assume the cortical bone edge in the image resides somewhere within the group of pixels. One such group of pixels in which the cortical bone edge lies in shown in chart 700 as pixels 123-125.

As mentioned above, the computing device may analyze the pixels of the range of pixels to determine the pixel with the lowest gray scale value. In one embodiment, the computing device may calculate the lowest gray scale value of the range of pixels where the pixel intensity can be approximately expressed as

I(x,yₘ)=p0m+p1mx+p2mx²+p3mx³+p4mx⁴,(x=n=n0,m0+1, . . . )

where the row index x assumes values x=1, 2, 3, . . . and the coefficients p0m, p1m, p2m, p3m, and p4m can be found by inversion of a 3×3 or 4×4 matrix involving powers of the pixel index numbers, n=n0, n0+1, n0+2, n0+3, n0+4. The 4^{th} degree of polynomial I(x,yₘ) in the equation may be less than 4 in appropriate circumstances. An approximation for a location of the “center” of the cortical bone can be estimated by a solution x=(x(min;m) (n0≦x(min)≦n0+4) of the equation

dI(x,yₘ)/dx=p1m+2p2mx+3p3mx²+4p4mx³=0.

The method for determining the cortical bone edge in the image of the patient's femur may also be utilized for other types of pixel ranges. As mentioned above, the pixel range may be a vertical range, or a number of pixels in the same column as the reference pixel. FIG. 8 is a screenshot of the MRI image 800 of the patient's femur with a selected point 802 and a vertical pixel range 804 around the selected point. Similar to the screenshot discussed above with reference to FIG. 6, the image 800 may include a reference point 802 associated with a reference pixel of the image. A range of pixels 804 may also be included oriented around the reference pixel. However, in this example, the range of pixels 804 forms a vertical column of pixels around the reference pixel. The orientation of the range of pixels 804 is just one example of the orientation of the range of pixels associated with the reference point.

In one embodiment, the orientation of the range of pixels may be known by the computing device when requesting the location of the reference point from the user of the computing device. For example, the computing device may request the user place the reference point in the image near a particular cortical edge of the bone of the image, such as the outer edge of the medial condyle of the femur in the image. Based on this request, the computing device may then create a horizontally-oriented range of pixels around the selected reference point to capture the cortical bone edge of the femur in the image. Similarly, the computing device may request the user place the reference point in the image near the most distal point of the femur in the image. Based on this request, the computing device may then create a vertically-oriented range of pixels around the selected reference point to capture the cortical bone edge of the femur in the image. In this manner, the computing device may request the placement of the reference point near a particular edge of the femur in the image and apply a range of pixels accordingly. In yet another embodiment, the computing device may analyze the image, select a particular reference point corresponding to a particular edge of the femur, and apply a particular orientation of a range of pixels around the reference point to attempt to capture the cortical bone edge of the femur in the image.

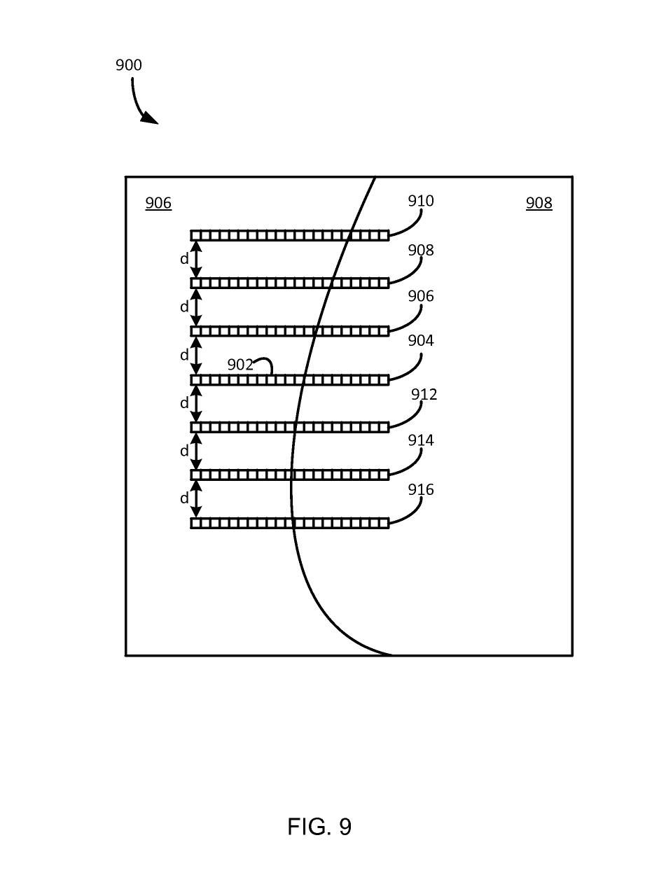

In addition, the computing device may also be configured to analyze several ranges of pixels in relation to determining the edge of the bone in an image based on a reference point in the image. For example, FIG. 9 is a screenshot of the MRI image of the patient's femur with a plurality of horizontal pixel ranges extending from a selected point by a set distance value. The image 900 is similar to the images described above with relation to FIG. 6 and FIG. 8. Also similar to the above description, the computing device may receive a reference point 902 from a user of the computer device or from an analysis of the image 900 by the computing device. A first range of pixels 904 may be created around the reference pixel 902 as described above and analyzed to determine a lowest gray scale value within the range of pixels. However, in addition to locating the pixel with the lowest gray scale value in the first range of pixels, the computing device may create additional ranges of pixels to further locate the cortical bone edge in the image.

In particular, the computing device may be configured to create additional ranges of pixels 906-916 in relation to the first range of pixels 904. For example, a second range of pixels 906 may be oriented a distance “d” from the first range of pixels in any direction. In the particular example illustrated in FIG. 9, the second range of pixels 906 is set off from the first range of pixels 904 vertically by the distance. As such, the second range of pixels 906 is oriented in a separate row of the image 900 from the first range of pixels 904. Upon the placement of the second range of pixels 906 in the image 900, the computing device may analyze the gray scale values of the pixels in the second range of pixels to determine the pixel or group of pixels with the lowest gray scale value. The edge of the cortical bone in the second range of pixels 906 may then be associated with the pixel with the lowest gray scale value. A third range of pixels 908 may then be created and placed in the image the distance d from the second range of pixels 906. The pixels of the third range of pixels 908 may be analyzed to determine the lowest gray scale value and the cortical bone edge within the third range of pixels.

In this manner, multiple ranges of pixels 904-916 may be created and analyzed to detect the edge of the cortical bone in the image 900. Further, the ranges of pixels 904-916 may be offset from each other by the distance d in any direction. For example, ranges of pixels 906-910 are oriented in rows above the reference point 902, while ranges of pixels 912-916 are oriented in ranges in rows below the reference point, with the distance between each range of pixels being the value d. Also, the placement of the ranges of pixels 904-916 may be in any direction from the reference pixel 902. Thus, ranges of pixels 904-916 may be horizontal or vertical from the first range of pixels 904. In addition, the ranges of pixels 904-916 may be in any orientation, such as vertical, horizontal, blocks, diagonal, etc. and may include more or fewer pixels than the first range of pixels 904. Finally, the distance between the ranges of pixels 904-916 may be any distance and may vary between the various ranges of pixels in the image 900. In this manner, the computing device may utilize pixel ranges 904-916 to locate the edge of the cortical bone in many locations within the image 900.

In one particular embodiment, the placement of the ranges of the pixels 904-916 may be adjusted upon the detection of the cortical bone edge in previous ranges of pixels. For example, upon the analysis of the second range of pixels 906 and the third range of pixels 908 in the image 900, the computing device may determine that the cortical bone is moving to the right within the ranges of pixels as the ranges of pixels are placed closer to the top of the image. In such a scenario, the computing device may begin orienting additional ranges of pixels to the right from the previous range of pixels. In this manner, the placement of the ranges of pixels may be adjusted as the cortical bone edge is determined through the analysis of previous ranges of pixels. In a similar manner, the orientation of the ranges of pixels may also be adjusted as the edge of the cortical bone is determined. In general, any configurable aspect of the range of pixels may be adjusted during the method described as more information about the location of the cortical bone edge is determined within the image.

Through the operations described above, a computing device may automatically determine or approximate the cortical bone or edge of the femur of a 2D image of a patient's knee joint. The location of the bone edge may aid a user of the computing device or the computing device itself in determining a cut plane for use in a TKA procedure of the patient's knee. For example, from the 2D images of the patient's knee and, in particular, one or more landmarks of the patient's knee identified in the 2D images, a cut plane through the patient's femur may be determined for use during a knee replacement procedure. The one or more landmarks may coincide with one or more edges of the patient's bone in the images. Thus, determining one or more edges of the patient's bone in the 2D images through the method described above may provide the one or more landmarks within the images to determine the cut plane used during the resection portion of the TKA. Because the method described above is more accurate than the user simply identifying the edge of the bone edge in the images through a computing device interface, the use of the method may provide a more accurate cut plane for use during the knee replacement procedure.

One example of the use of the edge detection of the patient's bone in one or more 2D images of the patient's joint is now described. In particular, a cut plane to resect a portion of a femur for use during a partial or total knee replacement procedure is provided. In general, the cut plane is determined from one or more landmarks or other portions of the patient's femur. Such landmarks may be identified in one or more image slices of the patient's knee and applied to the cut plane orientation. In one particular embodiment, the cut plane may be imported into a customized cutting jig for use during the TKA procedure. In general, during a TKA procedure, portions of the distal end of the femur (such as that shown in FIG. 2) are removed by the surgeon and replaced with an implant that approximates the shape and function of the ends of the respective bones. To aid in resecting portions of the femur and tibia, the surgeon may employ a femur cutting jig and tibia cutting jig that provides a cut or resection line for the surgeon to cut along.

To utilize the cut plane, the computing device may receive the 2D images or image slices of the joint from an imaging device and create a customized jig template from the images. Once the template for the cutting jig is created by the computing device utilizing one or more of the landmarks on the 2D images, a cutting or milling program is generated by the computing device. The cutting or milling program may then be provided to a milling machine to create the cutting jig corresponding to the milling program. The cutting jig is thus customized to the landmarks identified in the series of 2D images of the patient's joint. Further, the procedure does not require the generation of a three-dimensional (3D) model of the patient's anatomy to create the customized nature of the cutting jig. Rather, by utilizing one or more mating shapes that contact the joint anatomy at particular contact points of the joint anatomy corresponding to the identified landmarks in the 2D images, the customization of the cutting jig is achieved. Further, because the procedure does not require the generation of a 3D model, the customized cutting jigs may be produced more quickly and efficiently than previous customization methods.

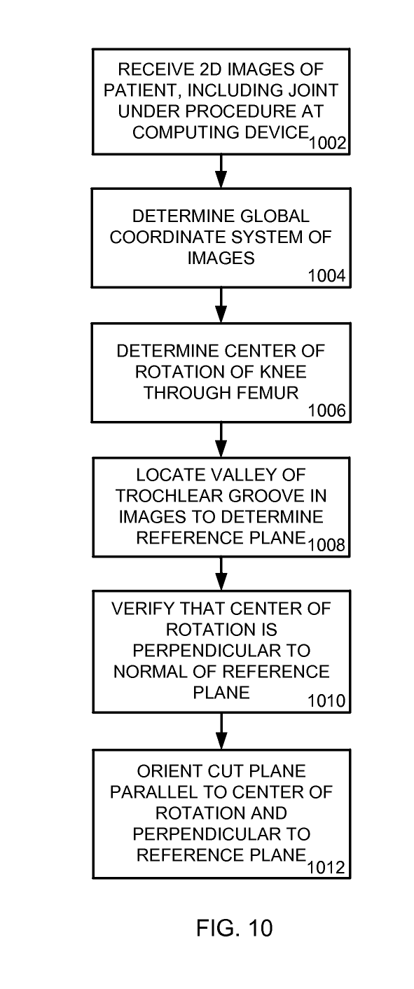

FIG. 10 is a flowchart illustrating a method for determining a cut plane for use during a knee replacement femur from one or more 2D images of the knee. The operations of the method of FIG. 10 may be performed by a computing device in operation by a user of the computing device. In addition, one or more of the operations may be performed by the computing device utilizing the cortical bone edge detection method discussed above. In general, the method provides an indication of a potential cut plane for use during a knee replacement procedure. Such a cut plane may be translated into a cutting jig for use during the procedure.

Beginning in operation 1002, a series of two-dimensional (2D) images of the patient's joint on which the arthroplasty procedure is to be performed may be obtained. The 2D images of the patient's joint may be obtained from an imaging device (such as an X-ray or magnetic resonance imaging (MRI) machine) from several aspects of the joint. Once the 2D images of the joint at issue are obtained, the images may be entered into a computing device for processing. The computing device may receive the images through any form of electronic communication with the imaging device. In one particular example, the 2D images may be obtained by the imaging device (such as the MRI imaging machine) and transmitted to a website accessible by the computing device. In general, however, the 2D images may be obtained from the imaging machine in any fashion for further processing by the computing device.

In operation 1004, the 2D images of the joint are processed to determine a global coordinate system for the images and/or to identify one or more points or landmarks associated with the patient's joint for establishing the cut plane. In general, a global coordinate system of the patient's joint in the images corresponds to the natural alignment of the patient prior to damage to the joint. For example, the global coordinate system of the images may correspond to an axial plane through the center of the patient's knee be parallel to the ground while the patient is walking. It should be appreciated, however, that reformatting the 2D images to achieve an image that is in anatomical alignment of the knee is not required. Rather, the reformatting of the images may approximate images of anatomical alignment of the knee for the global coordinate system.

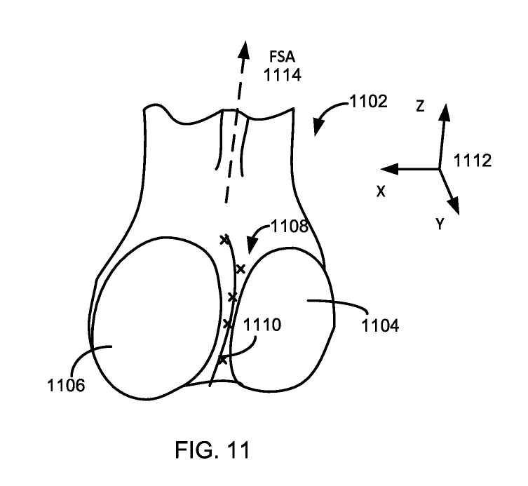

FIG. 11 is an illustration of a perspective anterior view lower portion of a femur shaft component. Portions of the femur 1102 model may be illustrated in the one or more image slices of the patient's femur mentioned above. That is, the femur 1102 may be a collection of the image slices of the patient's femur such that portions of the femur of FIG. 11 correspond to portions of the patient's femur, as provided in the received 2D images. In particular, the femur 1102 includes a medial condyle 1104 (located closes to a center of the body), an adjacent lateral condyle 1106, and a trochlear groove 1108 located between and defined by the medial condyle and the lateral condyle. The trochlear groove 1108 defines a valley along the surface of the femur 1102, with points on the floor of the valley 1110 lying approximately in a plane. This plane may be used in determining the cut plane for a resection of the femur during a TKA procedure, as explained in more detail below.

Also included in FIG. 11 is a global coordinate axis 1112. In general, the global coordinate system 1112 includes an x-axis, y-axis, and a z-axis. In one particular embodiment, the z-axis coincides with, or is approximately parallel to, a direction of a line segment that extends between a low end of the femur shaft to a high end of the femur shaft, or a femoral shaft axis 1114. The femur 1102 may be approximately straight or may be curvilinear such that the femoral shaft axis 1114 is generally linear through the femoral shaft. Also, the y-axis of the global coordinate system 1112 may extend in a direction (such as in the sagittal view) corresponding to a coronal view of the knee, and the x-axis is perpendicular to the y-axis and the z-axis.

In one particular embodiment, one or more of the 2D images may be reformatted along the global coordinate system. For example, the reformatting of the images may include reorientation of the images and/or extrapolation of data from between image slices to align or approximate the global coordinate system. Thus, each of the 2D images in the set of images may be reformatted to account for the angle of the images obtained during imaging. In one embodiment, one or more reference lines or points within the images may be analyzed when reorienting or reformatting the images along the global coordinate system. Such reference points or reference lines may be obtained through the operations described above to locate the edge of the femur bone in the images provided to the computing device. In yet another embodiment, the global coordinate system may be determined by the computing device in relation to the image or images with no additional formatting of the images occurring.

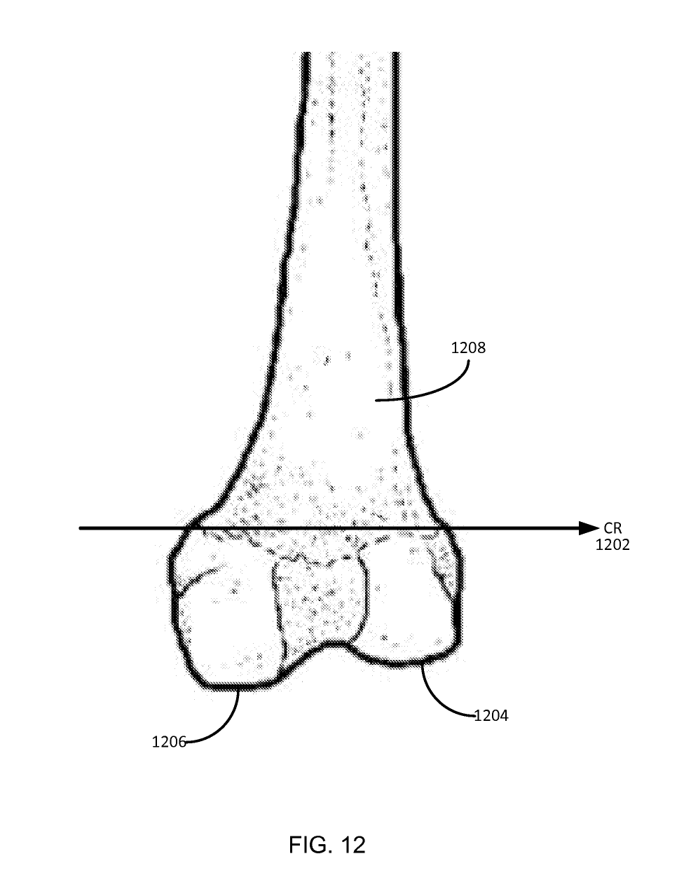

Once the global coordinate system of the images is determine, the computing device may determine a center of rotation of the femur in operation 1006 of FIG. 10. In general, the center of rotation of the knee is the center around which the patella, femur, and tibia rotate during extension and flexing of the joint. More particularly, when a knee tibia rotates relative to the corresponding knee femur, the patella moves along the trochlear groove and also rotates by a corresponding proportional amount, for example, by approximately the same rotation angle. Previously, it has been assumed that the center of rotation of the knee is focused on an approximate center of rotation of the tibia and the corresponding femur, which is often located within one or both knee condyles. However, by considering the center of rotation of the trochlear groove, the femur, and the tibia, a different center of rotation may emerge. Rotation of these three components by approximately the same angle indicates that these three components may have an approximate common rotation center, denoted CR 1202 and shown in FIG. 12 near the posterior edge of the femur at the base of the condyles 1204, 1206. Analysis of the knee indicates that points on the femur near the center of rotation 1202 move relatively little, or not at all, when the tibia, femur, and trochlear groove rotate relative to each other. As such, the center of rotation 1202 used herein is located near the posterior base of the condyles 1204, 1206 of the femur 1208, as indicated in one or more 2D images of a patient's knee.

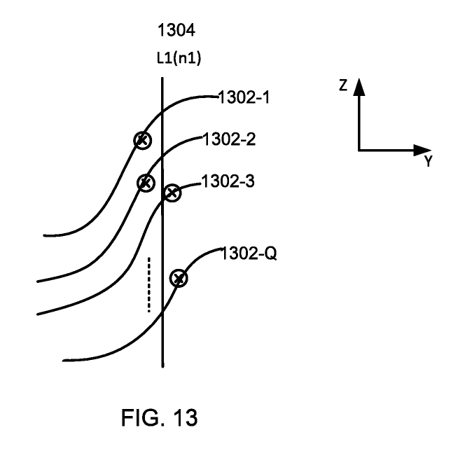

To determine the center of rotation 1202, one or more landmarks on the femur as represented in the 2D images of the patient's knee may be noted. In particular, FIG. 13 illustrates a portion of a surface of the femur corresponding to the base of the condyles on the posterior of the femur. In particular, the curved lines (1302-1 through 1302-Q) in the illustration represent the curvature of the portion of the femur corresponding to the base of the condyles on the posterior of the femur as seen in sagittal view through multiple image slices through the femur. For example, curved line 1302-1 may represent the portion of the femur corresponding to the posterior base of a medial condyle of the knee of the image while curved line 1302-Q represents the portion of the femur corresponding to the posterior base of the a lateral condyle of the knee of the image. As such, by traversing through the sagittal images of the femur represented in FIG. 13, an indication of the curved surface of the posterior base of the condyles of the femur illustrated in the image may be determined.

To create the curved lines (1302-1 through 1302-Q) of FIG. 13 of the posterior base of the condyles of the femur of the images, the computing device may perform one or more of the operations described above to locate the bone edge in the images. For example, the computing device may analyze a first sagittal image slice of the knee and, utilizing the methods described above, determine a curved line that represents the shape of the posterior base of the condyle for that particular slice. This curved line of the portion of the femur obtained through the bone edge detection described above may then be analyzed to determine the inflection point of the line as described in more detail below. Additional sagittal view image slices may similarly be analyzed to create the curved line that represents that portion of the femur in the images. In this manner, the curved lines (1302-1 through 1302-Q) of FIG. 13 of the posterior base of the condyles of the femur of multiple images of the patient's femur may be determined for additional analysis.

Once the curved lines (1302-1 through 1302-Q) are determined, the computing device may identify the inflection point of each curved line. Each of the inflection points corresponds to a point on the posterior base of the condyles of the femur in the 2D images. With the points on the femur identified, the computing device may calculate a best fit line that passes through or is adjacent each of the identified points along the posterior base of the condyles in the images. This best fit line is illustrated in FIG. 13 as line L1 1304. Line L1 1304 may correspond to a center of rotation line of the femur in the images that may be utilized by the computing device to determine a cut plane for use during resection of the femur of a TKA procedure. Thus, by locating the reference points in the 2D images, the center of rotation of the patient's knee may be determined.

One method to determine the center of rotation from the images is now presented. In FIG. 13, each curve (1302-1 through 1302-Q) lies in two coordinates (y coordinate and z coordinate) that can be approximated by a polynomial, z(y), for example, a cubic polynomial, z(y)=z0+z1y+z2y²+z3y³, or a higher degree polynomial in the coordinate y. An inflection point (y=yᵢ(q), z=zᵢ(q)) is defined herein as an estimated point where d²z/dy²=0 for the curve. A Best Fit linear or curvilinear line segment L1 1304 with an associated directional vector n1 (|n1|=1), illustrated in FIG. 13, can be determined or estimated that passes through or adjacent to all of the inflection points (yᵢ(q),zᵢ(q)). In general, each of the curves 1302-1 through 1302-Q will have a separate inflection point associated with that curve. Also, line L1 1304 may be considered the center of rotation of the knee and is referred to herein as such.

More particularly, each of these curves 1302-1 through 1302-Q can be approximately represented as a polynomial (e.g., cubic) curve

z(y;m)=a₀ₘ+a₁ₘy+a₂ₘy²+a₃ₘy³(m=1, . . . ,M),

where the polynomial coefficients, generally different for each curve (m), can be estimated by 3×3 or 4×4 matrix inversion. Each of these curves z(y;m) has an associated inflection point (d²z/d²y=0), with corresponding inflection point coordinates (y_{Im},z_{Im}) determined by 2a₂ₘ+6a₃ₘyₘₗ=0. A linear or nonlinear (e.g., quadratic) Best Fit curve is found for the coefficients a₀ₘ, a₁ₘ, a₂ₘ and a₃ₘ, using a least squares error

$$ɛ = {\sum }_{m = 1}^{M} {\{ {a}_{0 m} , + {a}_{1 m} {y}_{m l} + {{a}_{2 m} ( {y}_{m l} )}^{2} + {{a}_{m} ( {y}_{m l} )}^{3} - z ( {y}_{m l} ; m ; meas ) \}}^{2} .$$

These coefficients may be used to estimate coordinates (y_{Im},z_{Im}) for an inflection point for a femur condyle curve for each MRI image. A collection {(y₁,z₁)} of the inflection point coordinates is then used to estimate a Best Fit linear or curvilinear segment for an inflection line or curve passing adjacent to the inflection points (y_{Im},z_{Im}) and an associated directional vector n1 1304, illustrated in FIG. 13. This first approach for estimating a Best Fit curve for an inflection line utilizes at least 4 measurements of location for each of three MRI images, in order to estimate the coefficients a₀ₘ, a₁ₘ, a₂ₘ, and a₃ₘ for each of the femur curves.

A second approach utilizes an observation that the geometric region on which the femur curve locations would be measured is approximately planar, with an approximate defining equation for the plane P(FCC)

xβx+yβy+zβz−p=0,

where βx, βy, and βz are direction cosines for a vector V normal to the plane P(FCC) and p=|V| is a length of the vector V that extends from the coordinate origin to the plane P(FCC). In this second approach, the femur curve coefficients a₀ₘ, a₁ₘ, a₂ₘ, and a₃ₘ and the inflection point coordinates (y_{I},z_{I}) for this single curve are estimated by a polynomial

z=p(y;m)=a₀ₘ+a₁ₘy+a₂ₘ(y)²+a₃ₘ(y)³.

For the other femur curves of interest, each corresponding to an MRI image index m′=m−k, m−(k−1), . . . , m−1, m+1, . . . , m+(k−1), m+k, the defining polynomial is taken as

z=p(y;m′)=p(y+kρ1;m)

where ρ1 is a selected numerical parameter (positive, negative or 0). The inflection point coordinates for the corresponding polynomial z=p(y;m′) are then predicted or estimated to be

(y_{I}(k),z_{I}(k))=(y_{I}(k=0)−kρ1;z_{I}).

More generally, if the femur condyle curves lie on a curvilinear surface S(FCC) that has a planar component and/or a curvilinear component, the defining polynomial is taken to be

z=p(y;m′)=p((y+kρ1)(x+(1−χ)(p²)^{k};m)),

where x and ρ1 are selected numerical parameters with 0≦χ≦1. The inflection point coordinates for the corresponding polynomial z=p(y′;m′) are then predicted to be

(y_{I}(k),z_{I}(k))=({yI(k=0)/(χ+(1−χ)(ρ²)^{k})}}−kρ1;z_{I}.

This second approach, if available, allows reasonably accurate prediction of coordinates of an inflection point for each of M−1 femur curves, corresponding to M−1 MRI images, after estimation of the inflection point coordinates (y_{I},z_{I}) for a femur curve for a single MRI image. A Best Fit inflection line or curve that passes adjacent to or through each of the inflection point locations is estimated as in the first approach.

Regardless of the method used to obtain the center of rotation line of the femur in the images, the center of rotation 1304 calculation may be utilized by the computing device to determine a cut plane for use during a TKA procedure of a patient's knee. Additionally, and as shown in operation 1008 of FIG. 10, the computing device may also determine a valley of the trochlear groove of the patient's femur in the images to determine a reference line or plane for further use in determining the cut plane. In particular, the computing device may determine a plurality of locations along the trochlear groove illustrated in the 2D images provided to the computing device. In one embodiment, the locations along the valley of the trochlear groove are provided to the computing device by a user of the device. In another embodiment, the computing device may utilize the operations described above to locate the edge of the cortical bone in one or more images of the patient's knee that correspond to the trochlear groove of the femur. With the points along the valley floor of the trochlear groove identified, the computing device may determine a best fit line or best fit plane that may be used as a reference line or reference plane for determining a cut plane for use during a TKA procedure of the patient's knee.

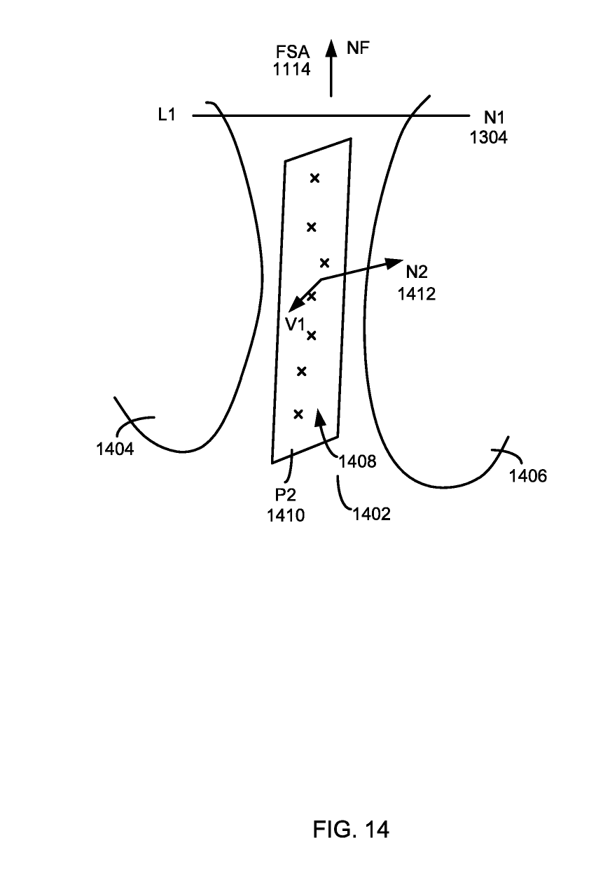

One particular method to determine the reference line or reference plane corresponding to the valley floor of the trochlear groove of the femur in the 2D images is now presented. In particular, FIG. 14 illustrates a condyle notch valley floor 1402 for a condyle notch in the images located between the two condyles 1404,1406 of the femur. The condyle notch valley floor 1108 is also illustrated in FIG. 11. Similar to the illustration of FIG. 11, the condyle notch valley floor 1402 of FIG. 14 includes selected points (“x”) 1408 on a curve representing the valley floor. As mentioned above, these selected points 1408 may be provided by a user of the computing device or may be determined by the computing device through the operations of edge detection disclosed above. In one particular embodiment, a series of coronal image slices of the patient's femur may be analyzed to determine the plurality of selected points 1408 along the condyle notch valley floor.

With the selected points 1408 noted in the images, the computing device may determine a Best Fit Plane or Best Fit Line P2 1410 in a similar manner used above to determine the center of rotation 1304. The Best Fit Line or Plane P2 1410 may have a directional vector n2 1412 (with magnitude |n2|=1), which can be estimated normal to the plane P2 that passes through or adjacent to the selected points on the valley floor 1402. In one particular implementation, an approach similar to the first approach discussed above is used to estimate locations on the trochlear groove valley floor 1402. A sequence of spaced apart MRI images in coronal view is formed, each such image is approximated by a quadratic or cubic curve, z=z(x), and location of a minimum valley floor error is estimated for each such curve. A Best Fit linear or curvilinear segment 1410 is estimated that passes adjacent to or through the locations of minimum valley floor height is estimated and used to determine a directional vector n2 1412 (|n2|=1). In other words, utilizing the equations discussed above, the direction cosines for the normal vector n2 1412 for the Best Fit Plane or Best Fit Line P2 1410 that passes through or adjacent to each of the designated locations on the condyle notch valley floor may be determined by the computing device. The normal vector n2 1412 to the plane P2 1410 may be utilized by the computing device in determining the cut plane for the TKA procedure, as explained in more detail below.

In operation 1010, the computing device may verify that the center of rotation 1304 determined above is perpendicular to the normal n2 1412 of the Best Fit Plane P2 1410 on the condyle notch valley floor 1402. If calculated correctly, then the center of rotation line 1304 (or vector n1), the normal to the reference plane 1412 (or normal n2), and the directional vector that is parallel to the femur shaft axis 1114 (or vector nF), all illustrated in relation to the femur of FIG. 14, may be utilized by the computing device to determine the cut plane for use in a TKA procedure in operation 1012. In general, the vector nF (parallel to the femur shaft axis discussed above) and at least one of the vectors, n1 and n2, may be used to estimate a direction of orientation of the femur cut plane in the TKA procedure. In one particular embodiment, the femur cut plane is generally parallel to the center of rotation (vector n1) and perpendicular to the reference plane such that the cut plane may be defined by the vector nF and at least one of the vectors n1 and n2, as vector n1 is perpendicular to vector n2.

In particular and returning to FIG. 14, a Best Fit line segment or Best Fit plane P2 1410 is shown as parallel to the inflection line L1 1304 determined above, a directional vector n2 1412 that is perpendicular to the Best Fit plane or line segment P2, and a directional vector nF 1114 that is parallel to the femur shaft axis. An angular orientation for a femur cut plane for a femur implant can be estimated by several approaches.

In a first approach, first and second intermediate vectors, v1 and v2, are defined,

v1=n1̂nF,

v2=v1̂n1=nF(n1·n1)−n1(nF·n1)=nF−n1(nF·n1),

where n1 1304 is the directional vector parallel to the inflection line L1 (preferably, with length |n1|=1). The vector v2 defines a normal vector direction for the femur cut plane and is perpendicular to n1 1304.

In a second approach, third and fourth intermediate vectors, are defined by

v3=n2̂nF,

v4=v3̂n2=nF(n2·n2)−n2(nF·n2)=nF−n2(nF·n2),

where the vector n1 is replaced by the vector n2(αx′,αy′,αz′). The vector v3 lies in the Best Fit plane or Best Fit line segment P2 1410 and is perpendicular to the directional vector n2 1412. The vector v4 has a major vector component parallel to the femur shaft axis 1114 and has a minor vector component, parallel to n2, that has a length (nF·n2), which is 0 if the directional vector n2 is perpendicular to the femur shaft axis directional vector nF; this occurs if nF lies in the plane P2 associated with the trochlear groove valley floor. The vector v4 provides a normal vector for orientation of normal vector for the femur cut plane with respect to the femur shaft axis 1114 or the central axis vector nF.

Based on the implant intended for the particular patient, a surgeon may select an appropriate distance from the distal point of the femur for positioning of the femur cut plane along the z-axis of the femur in the global coordinate system. However, through the operations above, the orientation of the cut plane, regardless of the selected distance from the distal point of the femur, may be determined from the 2D images provided to the computing device. In this manner, various landmarks of the patient's femur illustrated in the 2D images of the patient's knee may be utilized to determine an orientation of a cut plane to be used during a TKA procedure. Such a cut plane may be determined without the need to model the patient's knee or otherwise create a 3D interpretation of the images. As such, through the operations described above, a cut plane for use in TKA procedure is determined from the plurality of 2D images of the patient's joint.

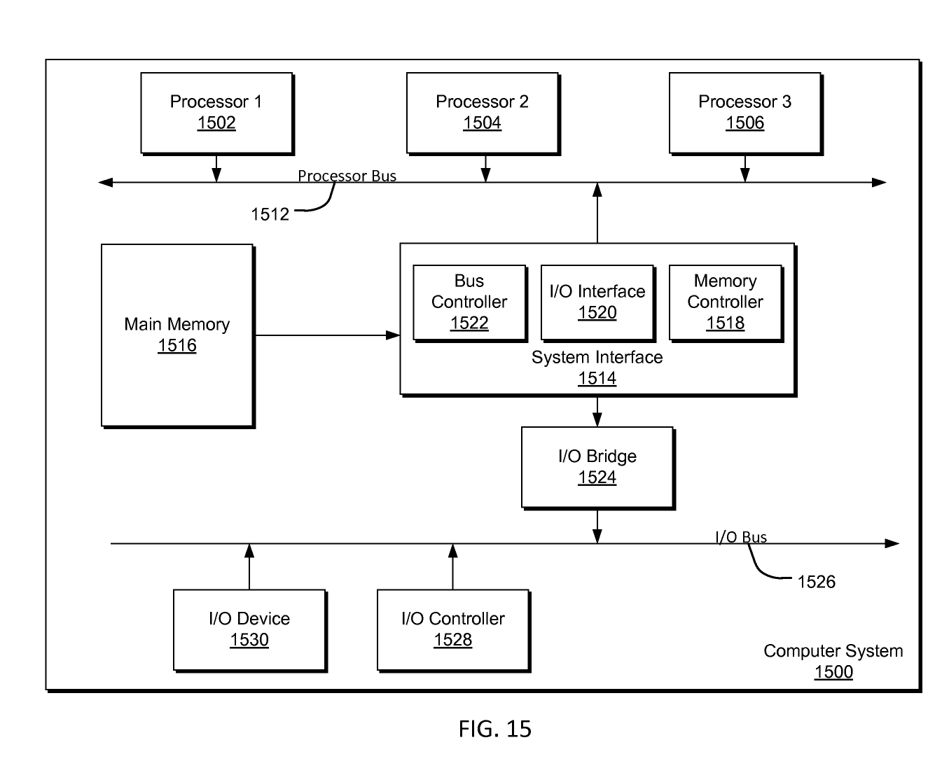

FIG. 15 is a block diagram illustrating an example of a computing device or computer system 1500 which may be used in implementing the embodiments disclosed above. The computer system (system) includes one or more processors 1502-1506. Processors 1502-1506 may include one or more internal levels of cache (not shown) and a bus controller or bus interface unit to direct interaction with the processor bus 1512. Processor bus 1512, also known as the host bus or the front side bus, may be used to couple the processors 1502-1506 with the system interface 1514. System interface 1514 may be connected to the processor bus 1512 to interface other components of the system 1500 with the processor bus 1512. For example, system interface 1514 may include a memory controller 1518 for interfacing a main memory 1516 with the processor bus 1512. The main memory 1516 typically includes one or more memory cards and a control circuit (not shown). System interface 1514 may also include an input/output (I/O) interface 1520 to interface one or more I/O bridges or I/O devices with the processor bus 1512. One or more I/O controllers and/or I/O devices may be connected with the I/O bus 1526, such as I/O controller 1528 and I/O device 1530, as illustrated.

I/O device 1530 may also include an input device (not shown), such as an alphanumeric input device, including alphanumeric and other keys for communicating information and/or command selections to the processors 1502-1506. Another type of user input device includes cursor control, such as a mouse, a trackball, or cursor direction keys for communicating direction information and command selections to the processors 1502-1506 and for controlling cursor movement on the display device.

System 1500 may include a dynamic storage device, referred to as main memory 1516, or a random access memory (RAM) or other computer-readable devices coupled to the processor bus 1512 for storing information and instructions to be executed by the processors 1502-1506. Main memory 1516 also may be used for storing temporary variables or other intermediate information during execution of instructions by the processors 1502-1506. System 1500 may include a read only memory (ROM) and/or other static storage device coupled to the processor bus 1512 for storing static information and instructions for the processors 1502-1506. The system set forth in FIG. 15 is but one possible example of a computer system that may employ or be configured in accordance with aspects of the present disclosure.

According to one embodiment, the above techniques may be performed by computer system 1500 in response to processor 1504 executing one or more sequences of one or more instructions contained in main memory 1516. These instructions may be read into main memory 1516 from another machine-readable medium, such as a storage device. Execution of the sequences of instructions contained in main memory 1516 may cause processors 1502-1506 to perform the process steps described herein. In alternative embodiments, circuitry may be used in place of or in combination with the software instructions. Thus, embodiments of the present disclosure may include both hardware and software components.

A machine readable medium includes any mechanism for storing or transmitting information in a form (e.g., software, processing application) readable by a machine (e.g., a computer). Such media may take the form of, but is not limited to, non-volatile media and volatile media. Non-volatile media includes optical or magnetic disks. Volatile media includes dynamic memory, such as main memory 1516. Common forms of machine-readable medium may include, but is not limited to, magnetic storage medium; optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions.

It should be noted that the flowcharts above are illustrative only. Alternative embodiments of the present invention may add operations, omit operations, or change the order of operations without affecting the spirit and scope of the present invention. The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

We claim:

1. A system for processing a medical scan of a patient, the system comprising:
a network interface configured to receive one or more medical images of a patient's anatomy;
a processing device in communication with the network interface; and
a computer-readable medium in communication with the processing device configured to store information and instructions that, when executed by the processing device, performs the operations of:
receiving the one or more medical images of the patient's anatomy from an imaging device;
obtaining a reference point on at least one of the medical images, the at least one medical image comprising a plurality of pixels;
associating the reference point to a reference pixel from the plurality of pixels of the at least one medical image;
creating a first range of pixels from the plurality of pixels of the at least one medical image corresponding to the reference pixel, wherein each pixel in the first range of pixels comprises a gray scale value;
determining the pixel in the first range of pixels with the lowest gray scale value; and
setting the determined pixel in the first range of pixels with the lowest gray scale value as a location of a bone edge in the at least one medical image.

2. The system of claim 1 wherein the one or more medical images are magnetic resonance imagining (MRI) images.

3. The system of claim 1 wherein the reference point is obtained as an input to the processing device from an input device, the input device operable by a user of the system.

4. The system of claim 1 wherein the first range of pixels comprises a group of pixels in a row of the plurality of pixels of the at least one medical image.

5. The system of claim 1 wherein the first range of pixels comprises a group of pixels in a column of the plurality of pixels of the at least one medical image.

6. The system of claim 1 wherein the information and instructions, when executed by the processing device, further performs the operation of creating a second range of pixels from the plurality of pixels of the at least one medical image and wherein the second range of pixels are different from the first range of pixels.

7. The system of claim 6 wherein the second range of pixels is offset from the first range of pixels in the at least one medical image by a distance.

8. The system of claim 7 wherein the information and instructions, when executed by the processing device, further performs the operations of:
creating a third range of pixels from the plurality of pixels of the at least one medical image, the third range of pixels different from the first range of pixels and the second range of pixels; and
offsetting the third range of pixels in the at least one medical image from the second range of pixels by the distance.

9. The system of claim 6 wherein the first range of pixels and the second range of pixels comprise a group of pixels in a row of the plurality of pixels of the at least one medical image, the row of the first range of pixels different than the row of the second range of pixels.

10. The system of claim 6 wherein the first range of pixels and the second range of pixels comprise a group of pixels in a column of the plurality of pixels of the at least one medical image, the column of the first range of pixels different than the column of the second range of pixels

11. A method for determining a cut plane through a human femur during an arthroplasty procedure on a human knee, the method comprising:
receiving a plurality of two-dimensional (2D) images of a patient's joint subject to the arthroplasty procedure at a computing device;
calculating a center of rotation of the human knee based at least on the one or more locations within at least one of the plurality of 2D images corresponding to a posterior base of a plurality of condyles of the human femur;
determining a best fit plane for a plurality of valley points along a valley floor of a trochlear groove of the human femur of a first set of 2D images of the plurality of 2D images;
calculating a cut plane for use during an arthroplasty procedure on a human knee, wherein the cut plane is parallel to the calculated center of rotation of the human knee and perpendicular to the best fit plane for the identified plurality of valley points along a valley floor of a trochlear groove of the human femur; and
generating a cutting jig for the arthroplasty procedure comprising a cut slot corresponding to the calculated cut plane.

12. The method of claim 11 further comprising identifying one or more locations within the at least one of the plurality of 2D images, the one or more locations corresponding to the posterior base of the plurality of condyles of the human femur.

13. The method of claim 11 further comprising identifying the plurality of valley points along the valley floor of the trochlear groove of the human femur in the first set of 2D images of the plurality of 2D images.

14. The method of claim 12 wherein the center of rotation comprises a best fit line calculation of the one or more locations corresponding to the posterior base of the plurality of condyles of the human femur.

15. The method of claim 11 further comprising determining a femoral axis line of the human femur within the at least one of the plurality of 2D images.

16. The method of claim 15 wherein the cut plane is calculated based at least on the femoral axis line and the center of rotation.

17. The method of claim 15 wherein the cut plane is calculated based at least on the femoral axis line and a calculated normal vector to the best fit plane.

18. The method of claim 11 wherein the calculated cut plane corresponds to a resection plane of the human femur during a total knee arthroplasty procedure.

19. A method for determining a resection plane of a femur of a human patient, the method comprising:
receiving a plurality of two-dimensional (2D) images of a patient's knee at a computing device from an imaging device, the 2D images comprising a femur of the patient's knee;
identifying one or more locations within at least one of the plurality of 2D images, the one or more locations corresponding to the posterior base of the plurality of condyles of the femur;
calculating a center of rotation axis of the patient's knee based at least on the one or more locations within at least one of the plurality of 2D images corresponding to a posterior base of a plurality of condyles of the human femur;
utilizing the center of rotation axis of the patient's knee to determine a cut plane through the femur during a resection of the femur for a arthroplasty procedure on the patient's knee; and
generating a cutting jig for the arthroplasty procedure comprising a cut slot corresponding to the cut plane.

20. The method of claim 19 further comprising:
obtaining a reference point on the at least one of the plurality of 2D images;
creating a range of pixels of the a at least one of the plurality of 2D images from the reference point, wherein each pixel in the range of pixels comprises a gray scale value; and
determining the pixel in the first range of pixels with the lowest gray scale value.
